(19) United States
(12) Patent Application Publication
(10) Pub. No.: US 2005/0054054 A1
(43) Pub. Date: Mar. 10, 2005

(54) Interleukin-7 molecules with altered biological properties

(76) Inventors: Francine M. Foss, Wellesley, MA (US); Larry Cosenza, Birmingham, AL (US)

Correspondence Address: FISH & RICHARDSON PC, 225 FRANKLIN ST, BOSTON, MA, 02110, US

(21) Appl. No.: 10/706,801

(22) Filed: Nov. 12, 2003

Related U.S. Application Data
(60) Provisional application No. 60/425,925, filed on Nov. 12, 2002.

(51) Int. Cl. C07H 21/04; C12P 21/04; C07K 14/54

(52) U.S. Cl. 435/69.52; 435/320.1; 435/325; 530/351; 536/23.5

(57) ABSTRACT

The present invention is based, in part, on the discovery that mutating IL-7 in the region of the carboxy terminus, can result in modification of receptor ligand interactions between IL-7 and IL-7 receptor (IL-7R). Compounds that modify such interaction, which are also within the scope of the invention, can be used to treat T-cell-mediated disorders.

### RELATED APPLICATIONS

This application claims the benefit of U.S. Provisional Application No. 60/425,925, filed Nov. 12, 2002, which is incorporated herein by reference in its entirety.

### TECHNICAL FIELD

This invention relates to cytokine-mediated therapies and therapeutics, and to IL-7-mediated therapies and therapeutics.

### BACKGROUND

An effective immune response begins when an antigen or mitogen triggers the activation of T cells. T cell activation is accompanied by numerous cellular changes, including the expression of cytokines and cytokine receptors. One of the cytokines involved in the immune response is interleukin-7 (IL-7), which acts as a differentiation and proliferation factor in B cells and a survival factor in activated T cells.

Receptors for IL-7 (IL-7R) have been found on cells of both the lymphoid and myeloid lineages. The heterodimeric IL-7R complex is composed of two subunits, a unique alpha (α) subunit and the p64 gamma (γ) subunit, which is common to high affinity isoforms of the IL-2, IL-4, IL-9 and IL-15 receptors. By way of the IL-7R, IL-7 induces cellular proliferation and differentiation by stimulating phoshoinositide turnover and through tyrosine phosphorylation events mediated by the Janus (Jak) and src-related kinases.

### SUMMARY

The present invention is based, in part, on the demonstration that an IL-7 mutant can function as a partial agonist of the IL-7 receptor (IL-7R) despite a lower binding affinity for that receptor. Accordingly, the invention encompasses IL-7 mutants (i.e., the polypeptides described below), nucleic acids that encode them, and cells and vectors that include those nucleic acids. The compositions of the invention have a variety of uses, including the treatment and diagnosis of proliferative disorders, including cancers, and other T-cell mediated processes such as transplant rejection (e.g., a graft rejection, such as an allograft rejection), and autoimmune disorders.

Provided herein are substantially pure polypeptides having an amino acid sequence that is identical to a wild type IL-7 sequence except for one or more amino acid residues in the carboxy-terminal helix D region. For example, the mutant IL-7 polypeptide can have a mutation in the region corresponding to amino acid positions 136-144 of SEQ ID NO:1 (the sequence of human IL-7) or in a corresponding region of an IL-7 polypeptide from another species (a non-human species). The mutation can be a deletion, addition, or substitution of one or more of the amino acids. If an IL-7 polypeptide has a substitution, for example, the substitution can be non-conservative, such as a substitution of a non-aromatic amino acid in the place of an aromatic amino acid. Alternatively, the substitution can be a conservative one.

An IL-7 polypeptide can have a mutation, such as a deletion, addition, or substitution, at the position corresponding to amino acid position 143 of SEQ ID NO:1. For example, an IL-7 polypeptide can have a substitution of the amino acid corresponding to position 143 of SEQ ID NO:1 with an alanine, proline, histidine, or tyrosine. Any of the mutant IL-7 polypeptides described herein may be able to compete effectively with wild type IL-7 for binding to a cell surface receptor, such as IL-7R.

An IL-7 polypeptide can include a heterologous amino acid sequence, and therefore be part of a chimeric polypeptide. The heterologous sequence can, in some cases, impart additional (e.g., beneficial) properties on the polypeptide. For example, a heterologous sequence may increase the circulating half-life of the chimeric polypeptide, or target the IL-7 polypeptide to a particular tissue or organ.

Isolated nucleic acid molecules encoding any of the IL-7 polypeptides described herein are provided, and expression vectors containing any of these nucleic acid molecules are also provided. An expression vector, for example, can include a sequence that encodes a detectable marker, such as green fluorescent protein (GFP), β-galactosidase, or chloramphenicol acetyl transferase. Alternatively, (or in addition), the detectable marker can be an epitope tag, such as a myc, FLAG, or HA tag. An expression vector can also include a gene encoding a selectable marker, such as a puromycin, neomycin, hygromycin, and/or ampicillin gene.

A cell containing any IL-7 polypeptide, nucleic acid molecule, or expression vector described herein is also provided.

An antibody, such as a monoclonal or polyclonal antibody, that specifically binds any of the described polypeptides, such as the mutant IL-7 polypeptides, is also provided.

Methods of treatment are provided. For example, the methods include treatment of a patient who has a proliferative disorder (such as a cancer (e.g., a leukemia, lymphoma, or myeloma)), an autoimmune disease, or a transplant rejection (e.g., a graft rejection). The methods include administering a composition, such as a pharmaceutical composition, that includes at least any one of the polypeptides, nucleic acid molecules, or expression vectors described herein. The amount of the composition administered to the patient is preferably sufficient to relieve at least some of the manifestations of the disease. For example, a patient who has a cancer can be administered an amount of the composition sufficient to inhibit the proliferation of the cancerous cells. The patient can be diagnosed with any one of a variety of cancers, including, but not limited to, an acute myelocytic leukemia, an adult acute lymphocytic leukemia, a childhood acute lymphocytic leukemia, a chronic lymphocytic leukemia, a chronic myelocytic leukemia, a hairy cell leukemia, Hodgkin's disease, a myelodysplastic syndrome, a non-Hodgkins lymphoma, an AIDS-related lymphoma, a cutaneous T-cell lymphoma, Sezary leukemia, an acute myelogenous leukemia, or a B cell chronic lymphocytic leukemia.

Also provided herein is a method of inhibiting the proliferation of a cell that expresses an IL-7 receptor. The method includes, for example, (a) providing a cell that expresses an IL-7 receptor, and (b) exposing the cell to a composition that contains any of the polypeptides, nucleic acids, or expression vectors described herein. The amount of the composition to which the cell is exposed, and the time of exposure, is preferably sufficient to inhibit the proliferation of the cell.

Also provided is a method of diagnosing a patient as having a disease or condition that is treatable with any of the IL-7 polypeptides, nucleic acids, or expression vectors described herein. The methods include determining whether a biological sample obtained from the patient contains cells that are bound by an IL-7 polypeptide or fragment thereof. If the patient contains such cells, then it can be determined that the cells can be bound in vivo by (and their proliferation subsequently inhibited by) any of the mutant IL-7 polypeptides described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. The materials, methods, and example are illustrative only and not intended to be limiting. Other features and advantages of the invention will be apparent from the accompanying drawings and description, and the claims. The contents of all references, pending patent applications and published patents, cited throughout this application are hereby expressly incorporated by reference. In case of conflict, the present specification, including definitions, will control.

### BRIEF DESCRIPTION OF THE DRAWINGS

FIG. 1A is a bar graph depicting the extent of cellular proliferation (Sezary leukemia cells) following exposure in culture to IL-2, IL-7, and two IL-7 mutants (IL-7W143A and IL-7W143H) (IL-7W143A indicates a substitution mutant substituting alanine in place of tryptophan at amino acid position 143; IL-7W143H indicates a substitution mutant substituting histidine in place of tryptophan at amino acid position 143), either alone or in various combinations. Cell proliferation was analyzed by [³H]-thymidine incorporation. The data represents percent of counts per minute (±SEM) of cells pulsed with ³H-TdR for the final 16 hours of culture.

FIG. 1B is a bar graph depicting the extent of cellular proliferation (B-CLL cells) following exposure in culture to IL-2, IL-7, and two IL-7 mutants (IL-7W143A and IL-7W143H), either alone or in various combinations. Cell proliferation was analyzed by [³H]-thymidine incorporation. The data represents percent of counts per minute (+SEM) of cells pulsed with ³H-TdR for the final 16 hours of culture.

FIG. 2 is a picture of the results of an assay that depicts Jak3 phosphorylation by IL-7 mutants. P116^{Jak3} was immunoprecipitated before and after exposure of HUT78 and NALM6 cells to IL-7 or an IL-7 mutant polypeptide. “A” indicates exposure to a IL-7W143A mutant polypeptide, and “H” indicates exposure to an IL-7W143H mutant polypeptide. Phosphorylation was measured by anti-phosphotyrosine immunoblotting (lanes marked “a”) and Coomassie blue staining of immunoblots (lanes marked “b”).

FIG. 3 is a picture of the results of gel assays depicting STAT phosphorylation in the presence of IL-7 mutant polypeptides. The polypeptides p86^{STAT3} and p85-p94^{STAT5} were immunoprecipitated after exposure of HUT78 and NALM6 cells to an IL-7 or an IL-7 mutant polypeptide. “A” indicates exposure to a IL-7W143A mutant polypeptide, and “H” indicates exposure to an IL-7W143H mutant polypeptide. Phosphorylation was measured by anti-phosphotyrosine immunoblotting (lanes marked “a”) and Coomassie blue staining of immunoblots (lanes marked “b”).

FIG. 4 is a picture of the results of gel assays depicting phosphorylation of src family kinases in the presence of IL-7 mutant polypeptides. The polypeptides p56^{lck}(3a) and p59^{fyn} (3b) were immunoprecipitated after exposure of HUT78 and NALM6 cells to an IL-7 or an IL-7 mutant polypeptide. Phosphorylation was measured by anti-phosphotyrosine immunoblotting (lanes marked “a”) and Coomassie blue staining of immunoblots (lanes marked “b”).

FIG. 5 is the wild type amino acid sequence of human IL-7.

FIG. 6 is a Table summarizing the effects on Jak3 and src kinases. Phosphorylation of Jak3 and src kinases was determined by immunoblot of immunoprecipitated proteins. Band intensities were compared using NIH image software. The results were demonstrated as fold differences at baseline of the unstimulated cells.

### DETAILED DESCRIPTION

This invention is based, at least in part, on the discovery that mutations in the carboxy terminus of IL-7 (e.g., one or more amino acids corresponding to positions 136-144 of SEQ ID NO:1), produce an IL-7 that differs from wild type IL-7 by, for example, having a different ability to interact with an IL-7 receptor (IL-7R) (e.g., the binding affinity or extent of signal transduction can vary between the mutant IL-7 and wild type IL-7).

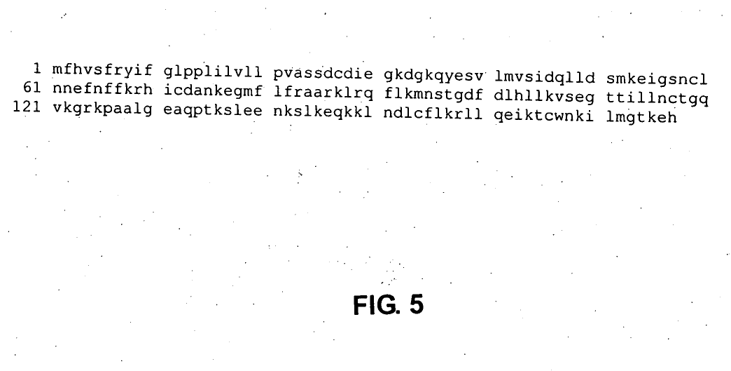

Structural modeling led to the prediction that the carboxy terminus of IL-7 is within a hydrophobic moment that is directed to a solvent interface. This suggests that the carboxy terminus is involved in protein-protein interactions (Cosenza et al., Protein Sci. 9:916-926, 2000; see also Cosenza et al., J. Biol. Chem. 272:32995-33000, 1997). As described in the example below, mutational analysis in this region of the carboxy-terminus identified IL-7R antagonists. More specifically, substitution of tryptophan at position 143 with an alanine or proline results in abrogation of IL-7-induced proliferation and alteration in tyrosine phosphorylation of signaling molecules in IL-7R-expressing human leukemia cells. Accordingly, IL-7 molecules containing one or more mutations within the carboxy terminal region are within the scope of the invention (particular IL-7 mutants include those having an addition, deletion, or substitution at amino acid position 143 of the human IL-7 sequence (corresponding to position 143 of SEQ ID NO:1) (see FIG. 5) or at analogous positions in IL-7 molecules of other species). As described herein, other residues can be mutated as well, and the invention encompasses mutant IL-7 polypeptides in which a single residue is changed (e.g., deleted or replaced with another residue), a pair of residues are changed (e.g., double mutants), or more than a pair of residues are changed (e.g., the mutant can be a triple mutant). The mutations can be of amino acid residues that are contiguous with one another, or the mutant residues may be separated by one or more wild type residues.

To determine whether any given IL-7 mutant has a biological activity that differs from wild type IL-7, one can assess the ability of the mutant to perform as wild type IL-7 would in the same circumstance. The following description illustrates the information available concerning IL-7 activity, and any of these activities (or others known in the art) can be assessed to determine whether a particular IL-7 mutant is an IL-7R antagonist and, therefore, a candidate therapeutic for treating diseases or disorders associated with IL-7-mediated cellular proliferation and other T-cell mediated processes (including, but not limited to, the cancers and autoimmune disorders described below), as well as transplant rejection, such as an allograft rejection).

IL-7 is a member of the type I cytokine group, which is identified primarily by a four α-helix bundle structure, the helices being designated as A, B, C, and D. The heterodimeric IL-7R complex is composed of two subunits, a unique alpha (a) subunit and the p64 gamma (γ) subunit, which is common to high affinity isoforms of the IL-2, IL-4, IL-7, IL-9 and IL-15 receptors (Goodwin et al., Cell 60:941-951, 1990; Noguchi et al., Science 262:1877-1880, 1993; see also Davies and Wlodawer, FASEB J. 9:50-56, 1995). Following IL-7R crosslinking, rapid activation of several kinases occurs, including members of the Janus and src families and P13-kinase (accordingly, one can assay kinase activation as a means of determining whether a mutant IL-7 binds to and antagonizes the IL-7R; a decrease in activation indicating a useful mutant and one that can be assessed further in cell-based assays in cell culture or in vivo as an IL-7R antagonist). A number of transcription factors are subsequently activated, including STATs, c-myc, NFAT and AP-1 (assays designed to evaluate these transcription factors can be used to assess any given IL-7 mutant; a modulation in activation can indicate a mutant that agonizes or antagonizes an IL-7R). Jak1 and P13 kinase are complexed to the intracytoplasmic domains of the IL-7Rα subunit, whereas Jak3 is complexed to the γc component, similar to IL-2R and IL-4R. Phosphorylation of both Jak1 and Jak3 initiate proliferation in activated T cells. The specific binding of IL-7 to the IL-7Rα subunit initiates heterodimerization with γc and phosphorylation of the Jak kinases. The Tyr residues in the cytoplasmic tail of the receptor thus provide docking sites for proteins with phosphotyrosine-binding SH2 domains, which in turn are also Jak substrates.

STAT3 and STAT5 have been shown to undergo phosphorylation upon ligand engagement of the IL-7R (Foxwell et al., Eur. J. Immunol. 25:3041-3046, 1995). In human peripheral blood T lymphoblasts, IL-2 and IL-7 were shown to be potentially equivalent in their ability to induce tyrosine phosphorylation of both STAT5 isoforms, STAT5a and STAT5b. The isoforms of STAT5 were shown to bind to related but distinct docking sites on the IL-7Rα chain. IL-7 induces STAT5a/STAT5b heterodimerization, and STAT3 seems to be associated constitutively with each STAT5 isoform. STAT1 is also activated upon stimulation of precursor B cells by IL-7.

IL-7R engagement also activates the src family kinases p59^{fyn} and p53^{lyn} in pre-B cells and in myeloid cell lines. In contrast to p53/p56^{lyn}, p59^{fyn} is associated constitutively with IL-7R in these cells. In mature human T cells, p56^{lck} was activated by IL-7 and IL-7R was distinctly shown to be physically associated with both p59^{fyn} and p56^{lck}. Signaling through p59^{fyn} is unlikely to mediate all of the responses generated by IL-7 (Hofineister et al., Cytokine Growth Factor Rev. 10: 14-60, 1999; Venkitaraman and Cowling, Eur. J. Immunol. 24:2168-2174, 1994).

Activated T-lymphocytes express high numbers of IL-7 receptors, and proliferation of the cells is driven by this receptor. T-cell activation is a process that occurs and leads to clinical symptoms and tissue damage in patients with autoimmune disorders or a transplant rejection (e.g., a graft rejection, such as an allograft rejection). Inhibition of the proliferation and signal transduction of these cells by IL7 mutants can decrease or eliminate symptoms of these diseases.

The polypeptides of the invention, which can be assessed in one or more of the assays described above, encompass substantially pure polypeptides having an amino acid sequence that is identical to a wild type IL-7 sequence (see FIG. 5) except that one or more amino acid residues in the carboxy-terminal helix D region is mutant. The polypeptides featured herein are conventional in that they can include amino acid residues (naturally occurring, synthetic, or modified (e.g., glycosylated or phosphorylated residues) residues) that are linked by a peptide bond.

In addition to containing one or more mutations, a polypeptide of the invention can be substantially pure (i.e., separated from one or more of the components that naturally accompany the polypeptide). Typically, a polypeptide is substantially pure when it is at least 60%, by weight, free from naturally occurring organic molecules. Alternatively, the preparation can be at least 75%, at least 90%, or at least 99%, by weight, mutant IL-7 polypeptide. A substantially pure mutant IL-7 polypeptide can be obtained, for example, by expression of a recombinant nucleic acid encoding a mutant IL-7 polypeptide, or by chemically synthesizing the polypeptide. Purity can be measured by any appropriate method, including column chromatography, polyacrylamide gel electrophoresis, or HPLC analysis. Polypeptides that are derived from eukaryotic organisms but synthesized in E. coli, or other prokaryotes, and polypeptides that are chemically synthesized will be substantially free from their naturally associated components.

A wildtype IL-7 can be a polypeptide that is identical to the naturally-occurring IL-7 polypeptide. IL-7 has been characterized functionally as a T cell growth factor that stimulates the proliferation and differentiation of B cells, T cells, natural killer (NK) cells, and lymphocyte-activated killer (LAK) cells in vitro.

A mutant polypeptide can be a polypeptide or portion thereof having at least one mutation relative to the wild-type molecule. A mutant IL-7 polypeptide that is biologically active generally modifies at least 40%, more preferably at least 70%, and most preferably at least 90% of the activity of the wild-type IL-7 molecule (for example, a mutant IL-7 polypeptide may bind an IL-7 receptor and reduce the proliferation of a population of receptor-bearing cells by about 40%, 50%, 60%, 70%, or more). The ability of a mutant IL-7 polypeptide to modify wild-type IL-7 activity can be assayed by numerous methods, including the cell proliferation and phosphorylation assays described herein.

A mutant IL-7 polypeptide can have a mutation in the region of amino acids 136-144 of, for example, the sequence shown in FIG. 5 (for example mutation of one or more of the amino acid residues at position 136, 137, 138, 139, 140, 141, 142, 143, or 144 of that sequence (the amino acid positions also correspond to the positions in SEQ ID NO:1)), or in a corresponding region of an IL-7 molecule from another species (for example, a domesticated animal such as a cow, pig, sheep, goat, rabbit, dog, or cat). Mutations within this region can be effected in any of the following ways: deletion of one or more of the amino acids, addition of one or more amino acids, or substitution of one or more of the amino acids.

In the event the mutation is a substitution, the substitution can be a conservative or non-conservative substitution. Non-conservative substitutions occur when one amino acid residue in a polypeptide sequence is replaced by another amino acid that has a different physical property (e.g., a different size, charge, or polarity) as the amino acid being replaced. For example, substitution of a non-aromatic amino acid in the place of an aromatic amino acid (e.g., substitution of an alanine in the place of tryptophan) is an example of a non-conservative substitution. Alternately, the substitution can be a conservative amino acid substitution. A conservative substitution can be the replacement of one amino acid in a polypeptide sequence by another amino acid, wherein the replacement amino acid has similar physical properties (e.g, size, charge, and polarity) as the amino acid being replaced. For example, replacing one aromatic amino acid with another aromatic amino acid can be a conservative substitution. Some typical examples of conservative amino acid substitutions include substitutions with the following groups: glycine and alanine; valine, isoleucine, and leucine; aspartic acid and glutamic acid; asparagine and glutamine; serine and threonine; lysine and arginine; and phenylalanine and tyrosine. The substitution can be, for example, a non-aromatic amino acid substitution.

A mutation in an IL-7 polypeptide can be a substitution of the amino acid at the position corresponding to position 143 of SEQ ID NO:1 (for example the sequence of human IL-7). The substitution can be for an alanine, proline, histidine, or tyrosine, for example. The substitution can also be at the corresponding position of an IL-7 polypeptide from another species (for example, a domesticated animal such as a cow, pig, sheep, rabbit, goat, dog or cat) and replacement with alanine, proline, histidine or tyrosine.

In some embodiments, the polypeptides described herein can effectively compete with wildtype IL-7 for binding to a cell surface receptor (for example, IL-7R). The polypeptides described herein can include a heterologous (i.e., non-IL-7) sequence (i.e., a polypeptide can be a chimeric polypeptide). For example, a heterologous amino acid sequence can increase the circulating half-life of the IL-7 portion of the polypeptide, such as albumin or the constant region of an immunoglobulin (e.g., an IgG).

The mutant IL-7 polypeptide, whether alone or as a part of a chimeric polypeptide, can be encoded by a nucleic acid molecule, and substantially pure nucleic acid molecules that encode the mutant IL-7 polypeptides described herein are within the scope of the invention. The nucleic acid can be a molecule of genomic DNA, cDNA, synthetic DNA, or RNA. The nucleic acid molecule encoding a mutant IL-7 polypeptide will be at least 65%, at least 75%, at least 85%, or at least 95% (e.g., 99%) identical to the nucleic acid encoding wild-type IL-7. For nucleic acids, the length of comparison sequences will generally be at least 50 nucleotides, preferably at least 60 nucleotides, more preferably at least 75 nucleotides, and most preferably 10 nucleotides.

The invention features isolated nucleic acid molecules having a sequence encoding any of the polypeptides described herein. The invention further features an expression vector having a nucleic acid molecule with a sequence encoding any of the polypeptides described above. The vector can be capable of directing expression of an IL-7 polypeptide in, for example, a cell that has been transduced with the vector. These vectors can be viral vectors, such as retroviral, adenoviral, or adenoviral-associated vectors, as well as plasmids or cosmids. Prokaryotic or eukaryotic cells that contain and express DNA encoding any of the mutant IL-7 polypeptides are also features of the invention. The method of transduction, the choice of expression vector, and the host cell may vary. The precise components of the expression system are not critical. It matters only that the components are compatible with one another, a determination that is well within the abilities of skilled artisans. Furthermore, for guidance in selecting an expression system, skilled artisans may consult Ausubel et al., Current Protocols in Molecular Biology (1993, John Wiley and Sons, New York, N.Y.) and Pouwels et al., Cloning Vectors: A Laboratory Manual (1987). The vector can also have a sequence that encodes a detectable marker, such as β-galactosidase, α-glucuronidase (GUS), luciferase, horseradish peroxidase (HRP), alkaline phosphatase, acetylcholinesterase, or chloramphenicol acetyl transferase. Fluorescent reporter genes include, but are not limited to, green fluorescent protein (GFP), red fluorescent protein (RFP), cyan fluorescent protein (CFP), and blue fluorescent protein (BFP). The detectable marker can also be an epitope tag, such as a myc, FLAG, or HA tag.

The present invention features a cell having any of the polypeptides described herein, any of the nucleic acid molecules described herein, or any of the expression vectors described herein (for example, a T cell or a B cell, in culture or in vivo). Also within the scope of the invention are antibodies (e.g., polyclonal or monoclonal antibodies) that specifically bind any of the polypeptides described herein. These antibodies can be made by methods known to those in the art of molecular biology and cellular biochemistry, and they can be used to detect the polypeptides of the invention in diagnostic or therapeutic contexts.

Antibodies An antibody that binds any of the polypeptides described herein is provided. A fragment of an antibody, such as an antigen-binding fragment, is also provided. The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat et al., Sequences of Proteins of Immunological Interest, Fifth Edition, U.S. Department of Health and Human Services, NIH Publication No. 91-3242, 1991; and Chothia et al., J. Mol. Biol. 196:901-917, 1987, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

An anti-IL-7 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen (e.g., the IL-7 mutant polypeptide or fragment thereof). Examples of antigen-binding fragments of an anti-IL-7 antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see Bird et al., Science 242:423-426, 1988; and Huston et al., Proc. Natl. Acad. Sci. USA 85:5879-5883, 1988). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

An anti-IL-7 antibody can be a polyclonal or a monoclonal antibody. The antibody can be recombinantly produced, for example, such as by phage display or by combinatorial methods.

Phage display and combinatorial methods for generating anti-IL-7 antibodies are known in the art (as described in Ladner et al. U.S. Pat. No. 5,223,409; Kang et al. International Publication No. WO 92/18619; Dower et al. International Publication No. WO91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. Bio/Technology 9:1370-1372, 1991; Hay et al., Hum. Antibod. Hybridomas 3:81-85, 1992; Huse et al., Science 246:1275-1281, 1989; Griffths et al., EMBO J. 12:725-734, 1993; Hawkins et al., J. Mol. Biol. 226:889-896, 1992; Clackson et al. Nature 352:624-628, 1991; Gram et al., PNAS 89:3576-3580, 1992; Garrad et al., Bio/Technology 9:1373-1377, 1991; Hoogenboom et al., Nuc. Acid Res. 19:4133-4137, 1991; and Barbas et al., PNAS 88:7978-7982, 1991, the contents of all of which are incorporated by reference herein).

An anti-IL-7 antibody can be a fully human antibody. For example, the antibody can be made in a mouse that has been genetically engineered to produce an antibody from a human immunoglobulin sequence. An anti-IL-7 antibody can also be a non-human antibody, such as a rodent (mouse or rat), goat, rabbit, primate (e.g., monkey), or camel antibody. Methods of producing antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, for example, Wood et al. International Application WO 91/00906, Kucherlapati et al. PCT publication WO 91/10741; Lonberg et al. International Application WO 92/03918; Kay et al. International Application 92/03917; Lonberg et al., Nature 368:856-859, 1994; Green et al., Nature. Genet. 7:13-21, 1994; Morrison et al., Proc. Natl. Acad. Sci. USA 81:6851-6855, 1994; Bruggeman et al., Year Immunol. 7:33-40, 1993; Tuaillon et al., PNAS 90:3720-3724, 1993; Bruggeman et al., Eur. J. Immunol. 21:1323-1326, 1991).

An anti-IL-7 antibody can be one in which the variable region, or a portion thereof, such as the CDR's, are generated in a non-human organism, such as a rat or mouse. Chimeric, CDR-grafted, and humanized antibodies are within the invention. Antibodies generated in a non-human organism, such as a rat or mouse, and then modified, such as in the variable framework or constant region, to decrease antigenicity in a human are also within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science 240:1041-1043, 1988; Liu et al., PNAS 84:3439-3443, 1987; Liu et al., J. Immunol. 139:3521-3526, 1987; Sun et al., PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988.

A CDR of an IL-7 antibody can be replaced with at least a portion of a non-human CDR or only some of the CDR's may be replaced with non-human CDR's. It is only necessary to replace the number of CDR's required for binding of the humanized antibody to a mutant IL-polypeptide or a fragment thereof.

A humanized or CDR-grafted antibody will have at least one or two but generally all three recipient CDR's (of heavy and or light immunoglobulin chains) replaced with a donor CDR. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

Methods of Treatment Methods are provided for treating a patient who has a T-cell-mediated disorder, such as a proliferative disorder (e.g, a cancer). A method can include administering to the patient a composition including any of the polypeptides described herein, any of the nucleic acids described herein (including an IL-7 polypeptide, IL-7R polypeptide, and any of the antibodies described herein), or any of the expression vectors described herein. The amount of the composition administered will be sufficient to inhibit the proliferation of cancerous cells in the patient. Moreover, these compositions can be administered together with (before, during, or after) other chemotherapies or radiation therapies. The cancer can be a leukemia, a lymphoma, or a myeloma. For example, the cancer can be an acute myelocytic leukemia, an adult acute lymphocytic leukemia, childhood acute lymphocytic leukemia, chronic lymphocytic leukemia, chronic myelocytic leukemia, hairy cell leukemia, Hodgkin's disease, a myelodysplastic syndrome, a non-Hodgkins lymphoma, an AIDS-related lymphoma, a cutaneous T-cell lymphoma, Sezary leukemia, an acute myelogenous leukemia, or B cell chronic lymphocytic leukemia.

Also, or in the alternative, methods are provided for treating a human having or at risk for having an autoimmune disorder. The methods include administering to the human a composition including any of the polypeptides described herein (including an IL-7 polypeptide and any of the antibodies described herein), any of the nucleic acids described herein, or any of the expression vectors described herein. The amount of the composition administered will be sufficient to inhibit the symptoms of the autoimmune disorder in the patient. Moreover, these compositions can be administered together with (before, during, or after) other therapeutic regimens (such as physical therapy, as for an arthritic condition, or extracorporeal photophoresis (ECP), such as in cases of GVHD). A human having or at risk for developing an autoimmune disorder can be diagnosed as having or at risk for developing (1) a rheumatic disease, such as rheumatoid arthritis, systemic lupus erythematosus, Sjbgren's syndrome, scieroderma, mixed connective tissue disease, dermatomyositis, polymyositis, Reiter's syndrome or Behcet's disease; (2) type I (insulin dependent) or type II diabetes mellitus; (3) an autoimmune disease of the thyroid, such as Hashimoto's thyroiditis or Graves' Disease; (4) an autoimmune disease of the central nervous system, such as multiple sclerosis, myasthenia gravis, or encephalomyelitis; (5) a variety of phemphigus, such as phemphigus vulgaris, phemphigus vegetans, phemphigus foliaceus, Senear-Usher syndrome, or Brazilian phemphigus; (6) psoriasis (e.g., psoriasis vulgaris) or atopic dermatitis; or (7) inflammatory bowel disease (e.g., ulcerative colitis or Crohn's Disease). The IL-7 (and IL-7R) polypeptides, nucleic acids, and vectors described herein can be used to treat other autoimmune disorders including, but not limited to, endogenous uveitis, nephrotic syndrome, primary biliary cirrhosis, lichen planus, pyoderma gangrenosum, alopecia greata, a Bullous disorder, chronic viral active hepatitis, autoimmune chronic active hepatitis, and acquired immune deficiency syndrome (AIDS).

Methods are also provided for treating a human having or at risk for developing an autoimmune disorder resulting from a transplant rejection, including an allograft (including xenograft) or autograft rejection, and including rejections of tissue, organ, or cell transplants. The disorder can be, for example, graft-versus-host-disease (GVHD), including acute or chronic GVHD, or aplastic anemia. The methods include administering to the human a composition including any of the polypeptides described herein (including an IL-7 polypeptide and any of the antibodies described herein), any of the nucleic acids described herein, or any of the expression vectors described herein. The amount of the composition administered will be sufficient to inhibit the symptoms of the transplant rejection in the human. For example, the human can reject a transplanted organ (such as a heart, liver, or kidney), a tissue graft (such as a skin graft), or a cell transplant (such as a bone marrow transplant). The treatment methods can improve or prevent any symptoms of a transplant rejection, including but not limited to symptoms associated with GVHD (acute or chronic GVHD). Moreover, these compositions can be administered together with (before, during, or after) other therapeutic regimens, such as extracorporeal photophoresis (ECP), as in cases of GVHD.

In therapeutic applications, the polypeptide can be administered with a physiologically-acceptable carrier, such as physiological saline by any standard route including intraperitoneally, intramuscularly, subcutaneously, or intravenously. It is expected that the intravenous route will be preferred. It is well known in the medical arts that dosages for any one patient depend on many factors, including the general health, sex, weight, body surface area, and age of the patient, as well as the particular compound to be administered, the time and route of administration, and other drugs being administered concurrently. Dosages for the polypeptides of the invention will vary, but a preferred dosage for intravenous administration is approximately 0.01 mg to 100 mg/ml blood volume. Determination of correct dosage for a given application is well within the abilities of one of ordinary skill in the art of pharmacology.

In addition, or in the alternative, methods are provided for inhibiting the proliferation of a cell that expresses an IL-7 receptor (for example, a lymphoid or a myeloid cell). The methods include providing a cell that expresses an IL-7 receptor and exposing the cell to a composition (for example, a pharmaceutical composition) having any of the polypeptides described herein, any of the nucleic acid molecules described herein, or any of the expression vectors described herein, wherein the amount of the composition to which the cell is exposed is sufficient to inhibit the proliferation of the cell. In addition, or in the alternative, methods are provided for diagnosing a patient as having a disease or condition that could be treated with any of the polypeptides described herein, any of the nucleic acids described herein, or any of the expression vectors described herein. The methods include determining whether a biological sample obtained from the patient contains cells that are bound by a polypeptide comprising IL-7, the occurrence of binding indicating that the cells can be bound by any of the polypeptides described herein in vivo and thereby inhibited from proliferating in response to wild-type IL-7 in vivo.

The mutant IL-7 polypeptide, either alone or as part of a chimeric polypeptide, can be encoded by a substantially pure nucleic acid molecule, including a molecule of genomic DNA, cDNA, or synthetic DNA. The nucleic acid molecule encoding a mutant IL-15 polypeptide will be at least 65%, at least 75%, at least 85%, or at least 95% (e.g., 99%) identical to the nucleic acid encoding wild-type IL-7. For nucleic acids, the length of comparison sequences will generally be at least 50 nucleotides, preferably at least 60 nucleotides, more preferably at least 75 nucleotides, and most preferably 110 nucleotides.

The DNA molecules described can be contained within a vector that is capable of directing expression of an IL-7 polypeptide in, for example, a cell that has been transduced with the vector. These vectors can be viral vectors, such as retroviral, adenoviral, or adenoviral-associated vectors, as well as plasmids or cosmids. Prokaryotic or eukaryotic cells that contain and express DNA encoding any of the mutant IL-7 polypeptides are also features of the invention. The method of transduction, the choice of expression vector, and the host cell may vary. The precise components of the expression system are not critical. It matters only that the components are compatible with one another, a determination that is well within the abilities of skilled artisans. Furthermore, for guidance in selecting an expression system, skilled artisans may consult Ausubel et al., Current Protocols in Molecular Biology (1993, John Wiley and Sons, New York, N.Y.) and Pouwels et al., Cloning Vectors: A Laboratory Manual (1987).

The invention is further illustrated by the following example, which should not be construed as further limiting.

### EXAMPLE

### IL-7 Mutants are Partial Agonists of the IL-7 Receptor

A series of IL-7 mutations in the region of amino acids 136-144 (of human IL-7) were prepared in an effort to determine the requirements for ligand-receptor interactions and to subsequently develop candidate receptor binding agonists and/or antagonists with selective biological activities. The IL-7 mutants were compared to each other and to native IL-7 with respect to their ability to induce cellular proliferation and to modulate signal transduction in neoplastic T cells and B cells.

IL-7W143A and IL-7W143H bind to IL-7R with less affinity (10-6 and 2×10⁻⁷ M respectively) compared to native IL-7 (IL-7W143) (2.5×10⁻⁸ M) (van der Spek et al., Cytokine 17:227-233, 2002). Both mutants, however, were capable of inducing proliferation of IL-7-dependent 2E8 cells (26% for IL-7W143H and 4% for IL-7W143A compared to native IL-7). To further examine the biological activity of the IL-7(143) mutants, we compared their ability to induce proliferation and signal transduction in neoplastic T cells and B cells. The following techniques were employed.

Normal lymphocytes and cell lines including pre-B leukemia cell Nalm-6 and cutaneous T cell lymphoma NCI-Hut 78 were obtained from the American Type Culture Collection. Fresh cells from patients with CTCL or B-CLL were also used in these studies.

Recombinant human IL-7 was obtained from the National Cancer Institute (NCI). IL-7W143A, IL-7W143F, IL-7W143H, IL-7W143P, and IL-7W143Y were kindly provided by Drs. Murphy and van der Spek (Boston University). The tryptophan (Trp, or W) residue normally present at position 143 was replaced in each case (van der Spek, supra). Any other mutation can be readily made by techniques practiced in the field of molecular biology.

For use in the experiments described below, an HRP-conjugated anti-human phosphotyrosine antibody (PY-20-HRPO) was obtained from Transduction Biotechnology (Lexington, Ky, USA). Polyclonal antihuman Jak3 antibody, anti-human STAT3, anti-human STAT5a and STAT5b antibodies were purchased from Santa Cruz Biotechnology, Inc. (Santa Cruz, Calif., USA).

Peripheral blood mononuclear cells (PBMC) were separated using Ficoll-hypaque (Pharmacia, Piscataway, N.J., USA) density gradient centrifugation. Adherent cells were removed by incubation of cells for 24 hours on culture flasks in complete medium (RPMI-1640 including 10% fetal bovine serum 50 μg/ml streptomycin and 50 μg/ml streptomycin and 50 μg/ml penicillin) at 5% CO₂ and 37° C.

Proliferation assays were performed by seeding cells in a 96-well flat-bottomed tissue culture plate (Costar, Cambridge, Mass., USA) at 5×10³ cells/100 μl in complete medium. Cells were treated with 150 U/ml IL-0.2, 10 ng/ml native rhIL-7 (obtained from NCI), IL-7W143A, IL-7W143F, IL-7W143H, IL-7W143P, IL-7W143Y and in combination with IL-2 respectively. The plates were incubated at 37° C., 5% CO₂ for 48 hours and 72 hours, pulsed with 1 μCi methyl-3H-TdR (specific activity is 0.25 mCi, 9250 KBQ, NEN, Life Science, Boston, Mass., USA), then harvested on fiber glass filters and thymidine incorporation was quantified by scintillation counting. All assays were performed in triplicate and results reported were ±SEM of triplicate assays.

Immunoprecipitation and Western blot analysis were performed as follows. Cell lines were cultured in tissue culture flasks until confluent prior to the experiment. Culture cells (5×10⁶ cells/ml) were serum starved for 4 hours and stimulated with 1 ml of serum-free medium containing 30 ng/ml rhIL-7 and IL-7W143 mutant forms for 2 minutes and 5 minutes. Cells were then lysed by the addition of lysis buffer (50 mM Tris-Cl, pH 8.2, 150 mM NaCl, 2 mM EDTA, 0.5% NP40, 1 mM sodium orthovandate, 10 μg/ml aprotinin, 10 μg/ml leupeptin, 5 mM sodium fluoride, 1 mM PMSF) on ice for 30 minutes. Cell lysates were collected and precipitated for 2 hours with preformed A/G sepharose (Sigma, St. Louis, USA) and primary antibody complex (Santa Cruz Biotechnology, Santa Cruz, Mass., USA) for overnight incubation at 4° C. Immunocomplexes were collected by centrifugation and washed three times in NP40 buffer (50 mM Tris-Cl, pH 8.2, 150 mM NaCl, 2 mM EDTA, 0.5% NP40). Reactions were terminated by the addition of SDS loading buffer. Samples were incubated in loading buffer for 15 minutes to remove protein from the beads, and the sepharose was removed by centrifugation. The supernatant was heated for 5 minutes prior to analysis by SDS-PAGE.

Immunoprecipitated proteins were separated by SDS-PAGE and transferred to polyvinylidene difluoride (PVDF) membranes. Protein blots were performed with antibody against human phosphotyrosine protein (PY-20-HRP conjugated). Immunoreactive bands were detected by Western blot chemiluminescence reagents (NEN Life Science, Boston, Mass., USA). Figures were scanned using HP-ScanJet 5370C and assembled using Adobe Photoshop. The density of phosphorylated protein bands was measured by NIH-image densitometer software.

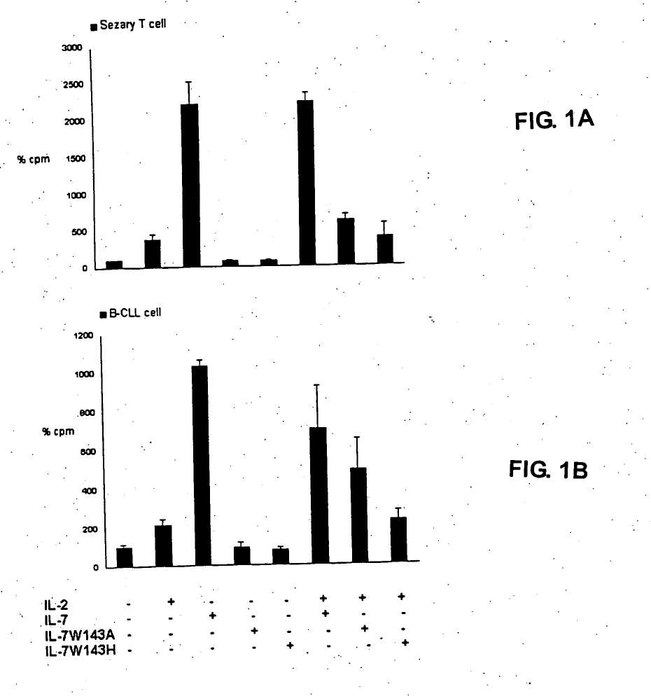

All of the IL-7 mutants induced less proliferation of the leukemia cells than native IL-7. The mutants without cyclic residues, IL-7W143A and IL-7W143P exhibited two-fold less IL-7 antagonist activity, whereas IL-7W143H and IL-7W143Y were more potent agonists. Neither IL-7W143A nor IL-7W143H were capable of stimulating proliferation of the Sezary cells when compared to native IL-7 (FIG. 1A). Further, the combination of either mutant with IL-2 induced less proliferation than native IL-7 and IL-2 in combination. The proliferation of the cells in the co-cultivation assays was similar to IL-2 alone, and there was no evidence of antagonism of IL-2-mediated proliferation in the presence of the mutants.

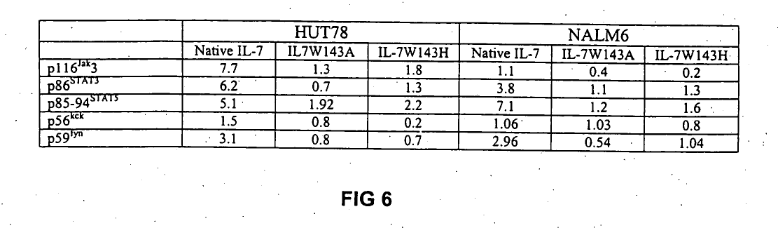

To determine whether the lack of proliferation in the presence of the IL-7 mutants was related to failure of the mutants to elicit IL-7R-mediated signaling events, tyrosine phosphorylation of p116^{Jak3}, p56^{lck and p}59^{fyn}, and the p85-96 STATs (STAT3, STAT5a, STAT5b) was analyzed by immunoprecipitation and anti-phosphotyrosine immunoblot (FIG. 6).

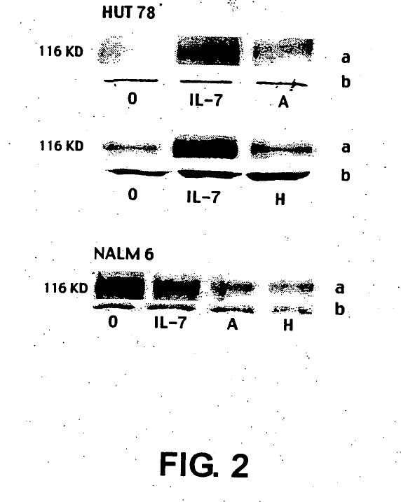

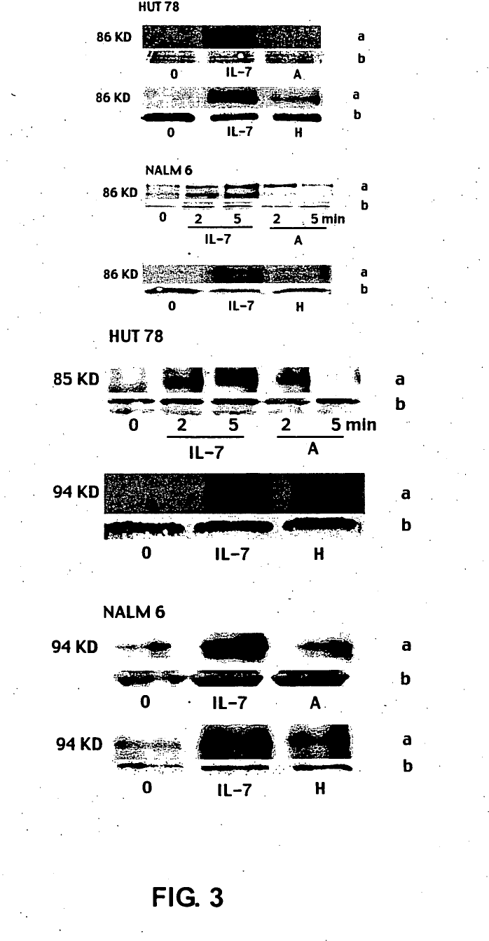

As shown in FIG. 2, IL-7 induces phosphorylation of p116^{Jak3} in the T cell leukemia cell line HUT78. Both IL-7W143A and IL-7W143H induced significantly less phosphorylation than native IL-7. In NALM6 cells, there was less phosphorylation in the presence of the IL-7 mutants suggesting that they may be competing with autophosphorylation induced by endogenous IL-7. Alteration in downstream signaling through STAT proteins was examined and paralleled the changes observed in Jak3 signaling with the mutants. There was a significant difference in phosphorylation of STAT3 by IL-7W143A and IL7W143H, but there appeared to be a more pronounced effect of IL-7W143A on STAT5, especially in the HUT78 cells (FIG. 3).

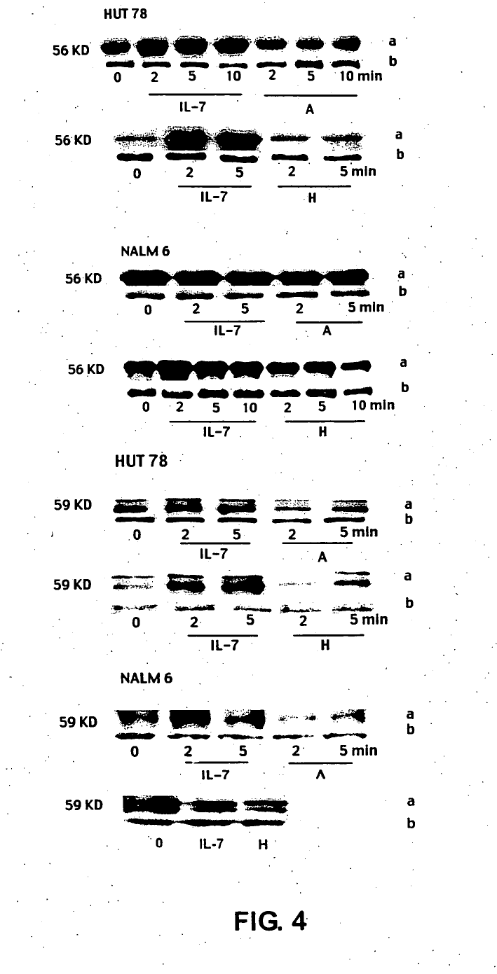

The effects of the IL-7 mutants on IL-7 mediated phosphorylation of p56^{lck} and p59^{fyn} are shown in FIG. 4. Phosphorylation or p56^{lck} was diminished in the presence of both mutants in the HUT78 cells and essentially unchanged in the NALM6 cells. p59^{fyn}, which 15′ has been shown to be complexed to the intracytoplasmic domain of the IL-7Rα subunit, was hypophosphorylated in both T and B cell lines.

Mutational studies of type I cytokines have identified three receptor binding sites on helices A and D and the long loops A-B and C-D (Campbell and Klinman, Eur. J. Immunol. 25:1573-1579, 1995). We recently proposed a model of hIL-7 that was constructed by comparative analysis, using the X-ray crystal structure of hIL-4 as a template (Bajorath et al., Protein Sci. 2:1798-1810, 1993). The model predicted that IL-7 exists as a four-helical bundle with an up-up-down-down topology. In this model, helix D is juxtaposed with helix A by disulfide bond assignment, and the predicted structure results in a net hydrophobic moment in helix D directed toward the solvent interface. Site-directed Ala substitution scanning mutagenesis in helix D resulted in the identification of a region between amino acids 136-144 which was important in receptor binding and bioactivity (Cosenza et al., supra). Substitution of tryptophan at position 143 with alanine or proline resulted in decreased proliferation of IL-7 dependent 2E8 cells, whereas substitution of other aromatic residues, including Phe or Tyr were indistinguishable from wild type, indicating that the presence of an aromatic residue at position 143 is required for biological activity. In these studies, His substitutions had intermediate binding and attenuated biological activity.

Since IL-7 has been shown to be both an autocrine and paracrine growth factor for human T and B leukemia cells, we compared the effects of the IL-7 mutants on proliferation of the cells. IL-7W143A and IL-7W143H induced less proliferation of the cells than native IL-7. The lack of antagonistic activity of the IL-7 mutants in the presence of IL-2 in co-cultivation assays suggests that there is no interaction between the IL-7 mutants and γc subunits, complexed with IL-2R subunits.

We demonstrated that both IL-7 mutants induced significantly less phosphorylation of Jak3 than native IL-7. In the case of IL-4, Kruse et al. (EMBO J. 11:3237-44, 1992) reported that substitutions of Tyr-124 in the C-terminal helix of IL-4 with Asp or Glu resulted in partial agonist/antagonist activity. One difference between these studies and our results is that IL-7W143A and IL-7W143H demonstrated a lower binding affinity to the IL-7R. Since Jak3 phosphorylation has been associated with engagement of γc, the abrogation of Jak3 signaling may be related, in part, to a disruption in the interaction between the hydrophobic residues in the carboxy terminus of the IL-7 mutants and γc. We demonstrate abrogation of 15′ Jak-related signaling but intact signaling through the src-related kinases, reiterating that the 11-7 mutants retain partial biological activity.

In summary, we demonstrated that IL-7W143A and IL-7W143H can function as partial agonists despite their lower binding affinities for the IL-7R. We demonstrated intact signaling via the src-related kinases p59^{fyn} and p56^{lck}, which have been shown to be physically associated with the p90 IL-7R (Seckinger and Fougereau, J. Immunol. 153:97-109, 1994; Page et al., Eur. J. Immunol. 25:2956-2960, 1995).

### Other Embodiments

A number of embodiments of the invention have been described. Nevertheless, it will be understood that various modifications may be made without departing from the spirit and scope of the invention. Accordingly, other embodiments are within the scope of the following claims.

1. A substantially pure polypeptide comprising an amino acid sequence that is identical to a wild type IL-7 sequence except that one or more amino acid residues in the carboxy-terminal helix D region is mutant.

2. The polypeptide of claim 1, wherein the polypeptide comprises a mutation in the region corresponding to amino acid positions 136-144 of SEQ ID NO:1 or in a corresponding region of an IL-7 polypeptide from another species.

3. The polypeptide of claim 2, wherein the mutation comprises a deletion of one or more of the amino acids corresponding to positions 136-144 of SEQ ID NO:1 or from a corresponding region of an IL-7 polypeptide from a non-human species.

4. The polypeptide of claim 2, wherein the mutation comprises an addition of one or more amino acids corresponding to positions 136-144 of SEQ ID NO:1 or to a corresponding region of an IL-7 polypeptide from a non-human species.

5. The polypeptide of claim 2, wherein the mutation comprises a substitution of one or more of the amino acids corresponding to positions 136-144 of SEQ ID NO:1 or in a corresponding region of an IL-7 polypeptide from a non-human species.

6. The polypeptide of claim 5, wherein the substitution comprises a non-conservative substitution.

7. The polypeptide of claim 5, wherein the substitution comprises substituting a non-aromatic amino acid in place of an aromatic amino acid.

8. The polypeptide of claim 2, wherein the mutation comprises a mutation at the position corresponding to position 143 of SEQ ID NO:1.

9. The polypeptide of claim 8, wherein the mutation comprises a substitution of the amino acid corresponding to position 143 of SEQ ID NO:1 with alanine or proline.

10. The polypeptide of claim 8, wherein the mutation comprises a substitution of the amino acid corresponding to position 143 of SEQ ID NO:1 with histidine or tyrosine.

11. The polypeptide of claim 5, wherein the substitution comprises a conservative substitution.

12. An isolated nucleic acid molecule comprising a sequence encoding a polypeptide of claim 1.

13. An expression vector comprising the nucleic acid molecule of claim 12.

14. The expression vector of claim 13, further comprising a sequence that encodes a detectable marker.

15. The expression vector of claim 14, wherein the detectable marker is a green fluorescent protein, β-galactosidase, or chloramphenicol acetyl transferase.

16. The expression vector of claim 14, wherein the detectable marker is an epitope tag.

17. A cell comprising the polypeptide of claim 1.

18. A cell comprising the nucleic acid molecule of claim 12.

19. A cell comprising the expression vector of claim 13.

20. An antibody that specifically binds the polypeptide of claim 1.

21. A method of treating a patient who has a T cell-mediated disorder, the method comprising administering to a patient a composition comprising a polypeptide of claim 1, and wherein the amount of the composition administered is sufficient to inhibit the symptoms of the T cell-mediated disorder in the patient.

22. A method of treating a patient who has a T cell-mediated disorder, the method comprising administering to a patient a composition comprising the nucleic acid molecule of claim 12, and wherein the amount of the composition administered is sufficient to inhibit the symptoms of the T cell-mediated disorder in the patient.

23. A method of treating a patient who has a T cell-mediated disorder, the method comprising administering to a patient a composition comprising the expression vector of claim 13, the amount of the composition administered being sufficient to inhibit the symptoms of the T cell-mediated disorder in the patient.

24. The method of claim 21, wherein the T-cell-mediated disorder is a cancer.

25. The method of claim 21, wherein the T-cell-mediated disorder is an autoimmune disorder.

26. The method of claim 21, wherein the T-cell-mediated disorder is a transplant rejection.

27. The method of claim 24, wherein the cancer is a leukemia, a lymphoma, or a myeloma.

28. The method of claim 24, wherein the cancer is an acute myelocytic leukemia, an adult acute lymphocytic leukemia, a childhood acute lymphocytic leukemia, a chronic lymphocytic leukemia, a chronic myelocytic leukemia, a hairy cell leukemia, Hodgkins disease, a myelodysplastic syndrome, a non-hodgkins lymphoma, an AIDS-related lymphoma, a cutaneous T-cell lymphoma, a Sezary leukemia, an acute myelogenous leukemia, or a B cell chronic lymphocytic leukemia.

29. A method of inhibiting the proliferation of a cell that expresses an IL-7 receptor, the method comprising
(a) providing a cell that expresses an IL-7 receptor, and
(b) exposing the cell to a composition comprising the polypeptide of claims 1, wherein the amount of the composition to which the cell is exposed is sufficient to inhibit the proliferation of the cell.

30. A method of diagnosing a patient as having a disease or condition that could be treated with a polypeptide of claims 1, the method comprising determining whether a biological sample obtained from the patient contains cells that are bound by a polypeptide comprising IL-7, the occurrence of binding indicating that the cells can be bound by the polypeptide of any of claims 1 in vivo and thereby inhibited from proliferating in response to wild-type IL-7 in vivo.

31. The polypeptide of claim 1, wherein the polypeptide effectively competes with wild type IL-7 for binding to a cell surface receptor.

32. The polypeptide of claim 1, wherein the polypeptide further comprises a heterologous sequence.

33. The polypeptide of claim 32, wherein the heterologous sequence comprises a sequence that increases the circulating half-life of the IL-7 portion of the polypeptide.


SEQUENCE LISTING (1 sequence)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH